(19) United States
(12) Patent Application Publication
(10) Pub. No.: US 2015/0230914 A1
(43) Pub. Date: Aug. 20, 2015

(54) METHOD FOR FORMING DUAL-LAYER COMPOSITE MATERIAL, DUAL-LAYER COMPOSITE MATERIAL THEREBY, BIO-MEDICAL EQUIPMENT CONTAINING THE DUAL-LAYER COMPOSITE MATERIAL

(71) Applicant: HAN BIOMEDICAL INC., Taipei City (TW)

(72) Inventors: Hsiao-Cheng YEN, Taipei City (TW); Chih-Long CHANG, Taipei City (TW)

(73) Assignee: HAN BIOMEDICAL INC., Taipei City (TW)

(21) Appl. No.: 14/622,195

(22) Filed: Feb. 13, 2015

Related U.S. Application Data
(60) Provisional application No. 61/940,257, filed on Feb. 14, 2014.

(30) Foreign Application Priority Data
Feb. 5, 2015 (TW) 104103831

(51) Int. Cl. A61F 2/04; A61F 2/02

(52) U.S. Cl. CPC A61F 2/04; A61F 2/02; A61F 2240/001; A61F 2230/0071; A61F 2230/0008; A61F 2230/0069; A61F 2230/0067; A61F 2250/0024; A61F 2210/0076; A61F 2210/0004

(57) ABSTRACT

The present disclosure provides a method for forming a dual-layer composite material, including: coating a barrier-film layer-forming material onto the surface of a porous scaffold layer to form a dual-layer intermediate product; and drying the dual-layer intermediate product to form a dual-layer composite material which includes the porous scaffold layer and a barrier film layer, wherein the porous scaffold layer and the barrier film layer are inseparable from each other.

### CROSS REFERENCE TO RELATED APPLICATION

This application claims the benefit of U.S. Provisional Application No. 61/940,257, filed on Feb. 14, 2014, the entirety of which is incorporated by reference herein.

The present application is based on, and claims priority from, Taiwan Application Serial Number 104103831, filed on Feb. 5, 2015, the disclosure of which is hereby incorporated by reference herein in its entirety.

### TECHNICAL FIELD

The present disclosure relates to a bio-medical material, and in particular it relates to a dual-layer composite material and the formation method thereof.

### BACKGROUND

Fibrous tissues which result in adhesion usually connect between two tissues, and usually cross space of a body cavity, such as the peritoneal cavity. Generally, the causes of postsurgical adhesion may include tissue incisions, handling of internal organs, drying out of internal organs and tissues, contact of internal tissues with foreign materials, such as gauze, surgical gloves, stitches, etc., and blood or blood clots that were not rinsed out during surgery.

Operations on the lower abdomen and pelvis include bowel and gynecological surgeries, and the risk of postsurgical adhesion is high. While the “connections” that result from such adhesion may not cause problems, they may cause tissues or organs at the two ends thereof to become twisted or pulled away from their normal positions. This can negatively affect their normal physiological functioning, and cause pain and medical complications.

Therefore, a novel anti-adhesion material which can be used to promote postsurgical wound healing is needed.

### SUMMARY

The present disclosure provides a method for forming a dual-layer composite material, comprising: coating a barrier-film layer-forming material onto a surface of a porous scaffold layer to form a dual-layer intermediate product; and drying the dual-layer intermediate product to form a dual-layer composite material which comprises the porous scaffold layer and a barrier film layer, wherein the porous scaffold layer and the barrier film layer are inseparable from each other, and wherein a method for forming the porous scaffold layer comprises: mixing at least one first biodegradable polymer with a first solvent under a stirring speed of 3500-12000 rpm, at about 4-10° C., for about 90-180 minutes, to form a slurry, wherein the at least one first biodegradable polymer is selected from a group consisting of collagen, gelatin, chitosan, and a combination of collagen, gelatin or chitosan, and hyaluronic acid; placing the slurry into a mold and freezing the slurry; performing a lyophilization procedure on the slurry to remove water molecules and obtain a scaffold body; and performing a vacuum heating procedure on the scaffold body to dehydrate and crosslink the scaffold body to form the porous scaffold layer. The method for forming the barrier-film layer-forming material comprises: mixing a second biodegradable polymer with a second solvent and then letting it stand to form a gel, wherein the second biodegradable polymer is selected from a group consisting of collagen, gelatin, chitosan and hyaluronic acid; and stirring the gel to a homogeneous stage to form the barrier-film layer-forming material.

The present disclosure also provides a dual-layer composite material which is formed by the method for forming the dual-layer composite material mentioned above.

The present disclosure further provides bio-medical equipment which comprises the dual-layer composite material formed by the method for forming the dual-layer composite material mentioned above.

The present disclosure also provides a dual-layer composite material, comprising: a porous scaffold layer having the effect of accelerating wound-healing and/or tissue regeneration; and a barrier film layer formed on a surface of the porous scaffold layer, having the effect of preventing tissue-adhesion, wherein the porous scaffold layer and the barrier film layer are inseparable from each other, and wherein porous scaffold layer is formed by at least one first biodegradable polymer, and the at least one first biodegradable polymer is selected from a group consisting of collagen, gelatin, chitosan, and a combination of collagen, gelatin or chitosan, and hyaluronic acid, and wherein the barrier film layer is formed by a second biodegradable polymer, and the second biodegradable polymer is selected from a group consisting of collagen, gelatin, chitosan and hyaluronic acid.

Moreover, the present disclosure provides bio-medical equipment comprising the foregoing dual-layer composite material.

A detailed description is given in the following embodiments with reference to the accompanying drawings.

### BRIEF DESCRIPTION OF DRAWINGS

The present disclosure can be more fully understood by reading the subsequent detailed description and examples with references made to the accompanying drawings, wherein:

FIG. 1 shows the structure of the dual-layer composite material of the present disclosure;

FIG. 2 shows the dual-layer composite material of the present disclosure in the form of a membrane;

FIG. 3 shows an oval-shaped embodiment for the dual-layer composite material of the present disclosure in the form of a 3-dimensional shape comprising a hollow part or channel with at least one opening;

FIG. 4 shows another oval-shaped embodiment for the dual-layer composite material of the present disclosure in the form of a 3-dimensional shape comprising a hollow part or channel with at least one opening;

FIGS. 5A and 5B show a tube-shaped embodiment for the dual-layer composite material of the present disclosure in the form of a 3-dimensional shape comprising a hollow part or channel with at least one opening, wherein FIG. 5A is a front view, and FIG. 5B is a cross-sectional view;

FIGS. 6A and 6B show another tube-shaped embodiment for the dual-layer composite material of the present disclosure in the form of a 3-dimensional shape comprising a hollow part or channel with at least one opening, wherein FIG. 6A is a front view, and FIG. 6B is a cross-sectional view;

FIGS. 7A to 7D show a funnel-shaped embodiment for the dual-layer composite material of the present disclosure in the form of a 3-dimensional shape comprising a hollow part or channel with at least one opening. FIG. 7A is a front view, FIG. 7B is a longitudinal section view, FIG. 7C is a top view and FIG. 7D is a bottom view; and

FIGS. 8A to 8D show another funnel-shaped embodiment for the dual-layer composite material of the present disclosure in the form of a 3-dimensional shape comprising a hollow part or channel with at least one opening. FIG. 8A is a front view, FIG. 8B is a longitudinal section view, FIG. 8C is a top view and FIG. 8D is a bottom view.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are shown schematically in order to simplify the drawing.

In one embodiment, the present disclosure provides a method for forming a dual-layer composite material that has the effect of accelerating wound-healing and/or tissue regeneration while at the same time preventing tissue-adhesion.

The method for forming a dual-layer composite material of the present disclosure may comprise the following steps, but is not limited thereto.

First, a barrier-film layer-forming material is coated onto a surface of a porous scaffold layer to form a dual-layer intermediate product.

Then, the dual-layer intermediate product is dried to form a dual-layer composite material which comprises the porous scaffold layer and a barrier film layer. The preceding dual-layer intermediate product may be dried at about 15-30° C., but is not limited thereto. In one embodiment, the dual-layer intermediate product may be dried at a room temperature. Moreover, the dual-layer intermediate product may be dried for about 12-24 hours. In one embodiment, the dual-layer intermediate product may be dried for about 24 hours. In one specific embodiment, the dual-layer intermediate product may be dried at a room temperature for about 24 hours to form the dual-layer composite material.

In the dual-layer composite material formed by the preceding method of the present disclosure, the porous scaffold layer has the effect of accelerating wound-healing and/or tissue regeneration and the barrier film layer has the effect of preventing tissue-adhesion, and the porous scaffold layer and the barrier film layer are inseparable from each other.

Furthermore, a method for forming the porous scaffold layer which is mentioned in the method for forming a dual-layer composite material of the present disclosure, may comprise, but is not limited to, the following steps.

First, at least one first biodegradable polymer is mixed with a first solvent at a low temperature, under a high stirring speed to form a slurry. The at least one first biodegradable polymer mentioned above may account for about 1.0-2.0 wt % of the slurry, but is not limited thereto.

In this mixing step, the at least one first biodegradable polymer may be mixed with the first solvent at about 4-10° C., such as 5° C., but is not limited thereto. Moreover, the at least one first biodegradable polymer may be mixed with the first solvent under a stirring speed of about 3500-12000 rpm, such as under a stirring speed of about 10500 rpm, but is not limited thereto. In addition, the at least one first biodegradable polymer may be mixed with the first solvent for about 90-180 minutes, such as 90 minutes, but is not limited thereto. In one embodiment, the at least one first biodegradable polymer is mixed with the first solvent under a stirring speed of 3500-12000 rpm, at about 4-10° C., for about 90-180 minutes, to form the slurry. Furthermore, in one specific embodiment, the at least one first biodegradable polymer is mixed with the first solvent under a stirring speed of 10500 rpm, at about 5° C., for about 90 minutes, to form the slurry.

Examples of at least one first biodegradable polymer which is suitable for being used in the method for forming the porous scaffold layer mentioned above may include, but are not limited to, collagen, gelatin, chitosan, a combination of collagen, gelatin or chitosan, and hyaluronic acid, and so on. In one embodiment, the at least one first biodegradable polymer may be collagen, such as type I collagen. Moreover, the at least one first biodegradable polymer may be a combination of collagen, gelatin or chitosan, and hyaluronic acid, such as a combination of collagen and hyaluronic acid.

In the embodiment in which the at least one first biodegradable polymer may be a combination of collagen, gelatin or chitosan, and hyaluronic acid, a weight ratio of the collagen, gelatin or chitosan to the hyaluronic acid may be about 90-99.9:10-0.1, but is not limited thereto. For example, a weight ratio of the collagen to the hyaluronic acid may be about 93-94:7-6.

In addition, in the embodiment, in which the at least one first biodegradable polymer may be a combination of collagen, gelatin or chitosan, and hyaluronic acid, before the step of mixing at least one first biodegradable polymer with a first solvent at a low temperature, under a high stirring speed, to form a slurry, the method for forming the porous scaffold layer may further comprise mixing the collagen, gelatin or chitosan with the first solvent at a low temperature, under a high stirring speed, previously, to form a mixture and then pouring a solution of the hyaluronic acid into the mixture, but is not limited thereto.

In one embodiment, the step of mixing the collagen, gelatin or chitosan with the first solvent at a low temperature, under a high stirring speed, previously may be, for example, mixing the collagen, gelatin or chitosan with the first solvent under a stirring speed of 3500-12000 rpm, at about 4-10° C., for about 90-180 minutes, previously, to form a mixture.

Furthermore, the solution of the hyaluronic acid may be formed by dissolving the hyaluronic acid in a solvent. Examples of solvent which is suitable for dissolving the hyaluronic acid may comprise water, acetic acid, isopropanol, etc. In one embodiment, the acetic acid is 0.05 M acetic acid.

In addition, the first solvent used in the method for forming the porous scaffold layer mentioned above may comprise water, isopropanol or acetic acid, but is not limited thereto. In one embodiment, the first solvent used in the method for forming the porous scaffold layer mentioned above is isopropanol, and the isopropanol may be about 10-20% isopropanol, such as 10% isopropanol, but is not limited thereto. In another embodiment, the first solvent used in the method for forming the porous scaffold layer mentioned above is acetic acid, and the acetic acid may be about 0.02-0.05 M acetic acid, such as 0.05 M acetic acid, but is not limited thereto.

After the at least one first biodegradable polymer is mixed with the first solvent to form the slurry, the formed slurry is placed into a mold and frozen. In this step, the slurry may be frozen at about −40 to −20° C., but is not limited thereto. In one embodiment, the slurry may be frozen at about −30° C.

Next, after the slurry is placed into a mold and frozen, a lyophilization procedure is performed on the slurry to remove water molecules and obtain a scaffold body. The pressure in the preceding lyophilization procedure is about 30-200 mTorr, such as about 200 mTorr, but is not limited thereto. Moreover, the lyophilization procedure may comprise at about −40 to −20° C., freezing the slurry for about 3-6 hours, then at about −40 to 10° C., freezing the slurry for about 12-16 hours, and after that placing the slurry at about 20 to 30° C., for about 3-6 hours, but is not limited thereto. In one embodiment, the pressure in the preceding lyophilization procedure may be 200 mTorr, and in this embodiment, the lyophilization procedure may comprise at about −30° C., freezing the slurry for about 3-6 hours, then at about 0° C., freezing the slurry for about 12-16 hours, and after that placing the slurry at about 30° C., for about 3-6 hours.

Furthermore, after the step of performing a lyophilization procedure on the slurry to remove water molecules and obtain a scaffold body, a vacuum heating procedure is performed on the obtained scaffold body to dehydrate and crosslink the scaffold body to form the porous scaffold layer mentioned above. The pressure in the vacuum heating procedure may be about 30-200 mTorr, such as about 200 mTorr, but is not limited thereto. In addition, the temperature in the vacuum heating procedure may be about 80-110° C., but is not limited thereto. Furthermore, time for the vacuum heating procedure may be about 12-24 hours, such as about 24 hours, but is not limited thereto. In one specific embodiment, the pressure in the vacuum heating procedure is about 200 mTorr, the temperature in the vacuum heating procedure is about 80-110° C., and time for the vacuum heating procedure is about 24 hours.

In addition, in one embodiment, the method for forming the porous scaffold layer in addition to the steps mentioned above, between the step of mixing at least one first biodegradable polymer with a first solvent at a low temperature, under a high stirring speed to form a slurry and the step of placing the slurry into a mold and freezing the slurry, may further comprise a step of removing gas in the slurry. There is no specific limitation for the manner for removing gas in the slurry. In one embodiment, gas in the slurry may be removed by vacuum heating.

Moreover, a method for forming the barrier-film layer-forming material which is mentioned in the method for forming a dual-layer composite material of the present disclosure, may comprise, but is not limited to, the following steps.

First, a second biodegradable polymer is mixed with a second solvent and then left to stand to form a gel. The second biodegradable polymer may account for about 3-10% w/v of the gel, but is not limited thereto.

Examples of second biodegradable polymer which is suitable for being used in the method for forming the barrier-film layer-forming material mentioned above may include, but are not limited to, collagen, gelatin, chitosan, hyaluronic acid, etc. In one embodiment, the second biodegradable polymer may be collagen, such as type I collagen.

In addition, the second solvent used in the method for forming the barrier-film layer-forming material mentioned above may comprise water or acetic acid, but is not limited thereto. In one embodiment, the second solvent used in the method for forming the porous scaffold layer mentioned above is acetic acid, and the acetic acid may be about 0.1-0.5 M acetic acid, such as 0.5 M acetic acid, but is not limited thereto.

Next, after forming the gel, the gel is stirred to a homogeneous stage to form the barrier-film layer-forming material.

Moreover, in one embodiment, the method for forming the barrier-film layer-forming material in addition to the steps mentioned above, after the step of stirring the gel to a homogeneous stage, may further comprise a step of removing bubbles in the gel.

In another embodiment, the present disclosure provides a dual-layer composite material, which is formed by any of the methods for forming a dual-layer composite material of the present disclosure, and the mentioned methods for forming a dual-layer composite material may also comprise various aforementioned methods for forming the porous scaffold layer and various aforementioned methods for forming the barrier-film layer-forming material.

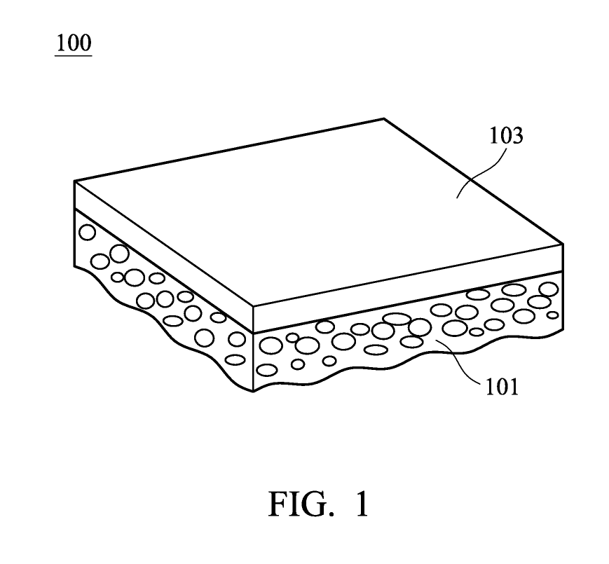

The dual-layer composite material formed by the method for forming a dual-layer composite material of the disclosure has a structure shown in FIG. 1. FIG. 1 shows that dual-layer composite material of the present disclosure 100 has a porous scaffold layer 101 and a barrier film layer 103, wherein the barrier film layer 103 is formed on a surface of the porous scaffold layer 101.

The dual-layer composite material of the present disclosure has the effect of accelerating wound-healing and/or tissue regeneration while at the same time preventing tissue-adhesion. The dual-layer composite material of the present disclosure can be applied in bio-medical use, for example, the dual-layer composite material of the present disclosure can be applied to postsurgical wounds, but is not limited thereto. Furthermore, the direction for use of the dual-layer composite material of the present disclosure is attaching the porous scaffold layer of the dual-layer composite material to a wound.

The porous scaffold layer of the dual-layer composite material of the present disclosure has the effect of accelerating wound-healing and/or tissue regeneration, and can be degraded naturally after the wound has healed. The porous scaffold layer mentioned above is a scaffold structure, and a pore size of the porous scaffold layer may be about 100-500 μm, but is not limited thereto.

The barrier film layer of the dual-layer composite material of the present disclosure has the effect of preventing tissue-adhesion. When the dual-layer composite material of the present disclosure is applied to a wound, the barrier film layer will become a gel within 24 to 48 hours, and can be slowly resorbed and excreted from the body in less than 28 days. The foregoing barrier film layer is a non-porous layer, and the barrier film layer has the property of separating from the injury site and the function of providing a proper interface between the wound and other areas, attenuating the inflammatory signaling, etc., but is not limited thereto.

The dual-layer composite material of the present disclosure may be in the form of a membrane or in the form of a 3-dimensional shape, but is not limited thereto.

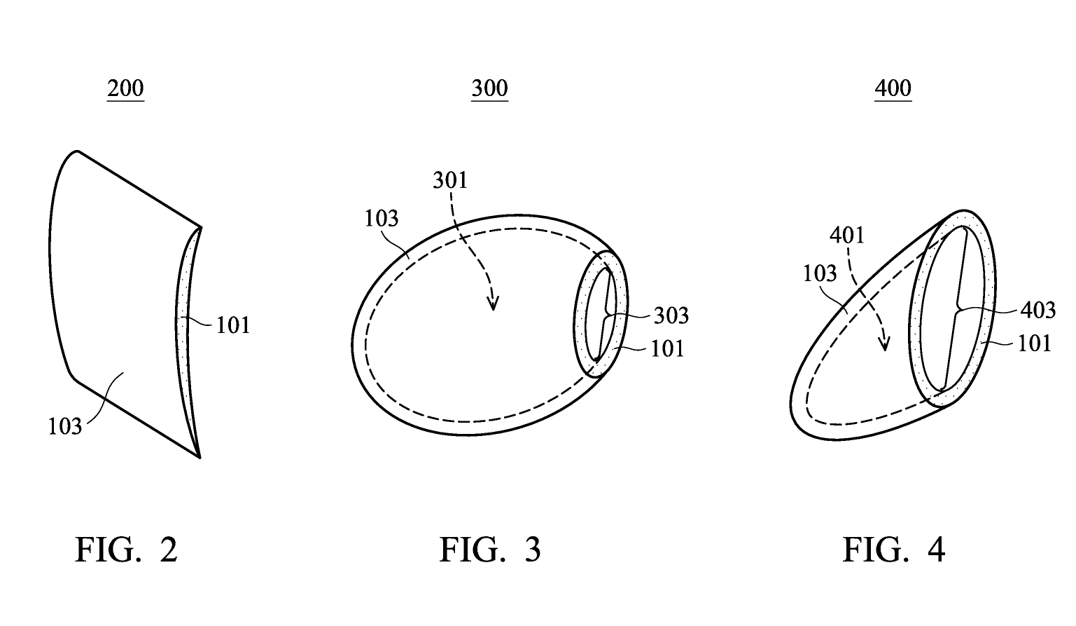

FIG. 2 shows the dual-layer composite material of the present disclosure 200 in the form of a membrane which has a porous scaffold layer 101 and a barrier film layer 103.

The dual-layer composite material of the present disclosure may comprise a 3-dimensional shape comprising a hollow part or channel with at least one opening, and the 3-dimensional shape may be for example, a globe shape, an oval-shape, a tube shape, a funnel shape, a cone shape, etc., but is not limited thereto. In one embodiment, in the dual-layer composite material of the present disclosure in the form of a 3-dimensional shape comprising a hollow part or channel with at least one opening, the porous scaffold layer is located outside of the 3-dimensional shape while the barrier film layer is located inside of the 3-dimensional shape and encompasses the hollow part or channel. In another embodiment, the barrier film layer is located outside of the 3-dimensional shape while the porous scaffold layer is located inside of the 3-dimensional shape and encompasses the hollow part or channel.

FIG. 3 shows an oval-shaped embodiment for the dual-layer composite material of the present disclosure in the form of a 3-dimensional shape comprising a hollow part or channel with at least one opening. In the oval-shaped dual-layer composite material comprising a hollow part or channel with at least one opening of the present disclosure 300 of FIG. 3, the barrier film layer 103 is located outside of the oval-shaped dual-layer composite material of the present disclosure while the porous scaffold layer 101 is located inside of the oval-shaped dual-layer composite material of the present disclosure 300 and encompasses the hollow part 301 and the opening 303.

FIG. 4 shows another oval-shaped embodiment for the dual-layer composite material of the present disclosure in the form of a 3-dimensional shape comprising a hollow part or channel with at least one opening. In the oval-shaped dual-layer composite material comprising a hollow part or channel with at least one opening of the present disclosure 400 of FIG. 4, the barrier film layer 103 is located outside of the oval-shaped dual-layer composite material of the present disclosure while the porous scaffold layer 101 is located inside of the oval-shaped dual-layer composite material of the present disclosure 400 and encompasses the hollow part 401 and the opening 403.

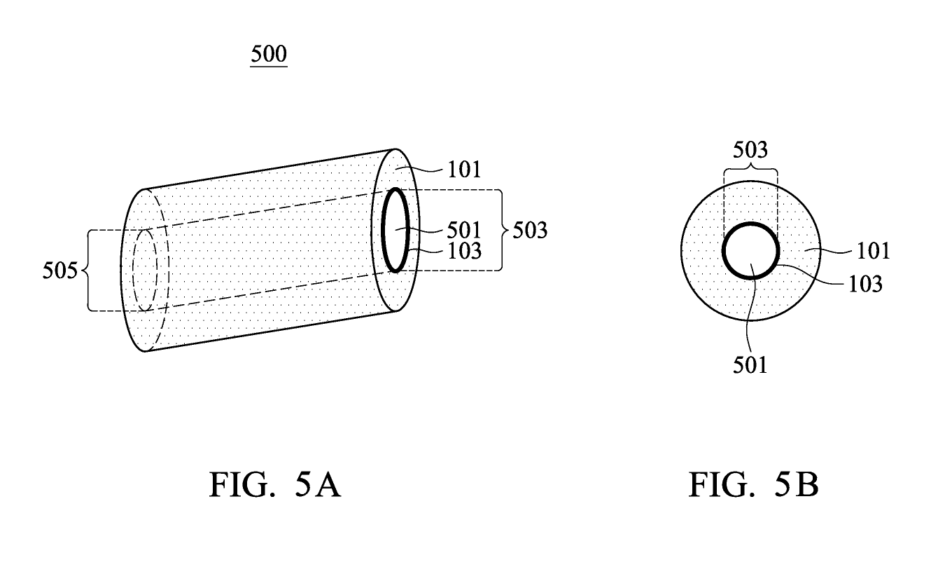

FIGS. 5A and 5B show a tube-shaped embodiment for the dual-layer composite material of the present disclosure in the form of a 3-dimensional shape comprising a hollow part or channel with at least one opening. FIG. 5A is a front view, and FIG. 5B is a cross-sectional view. In the tube-shaped dual-layer composite material comprising a hollow part or channel with at least one opening of the present disclosure 500 of FIGS. 5A and 5B, the porous scaffold layer 101 is located outside of the tube-shaped dual-layer composite material of the present disclosure while the barrier film layer 103 is located inside of the tube-shaped dual-layer composite material of the present disclosure 500 and encompasses the channel 501, the opening 503, and the opening 505.

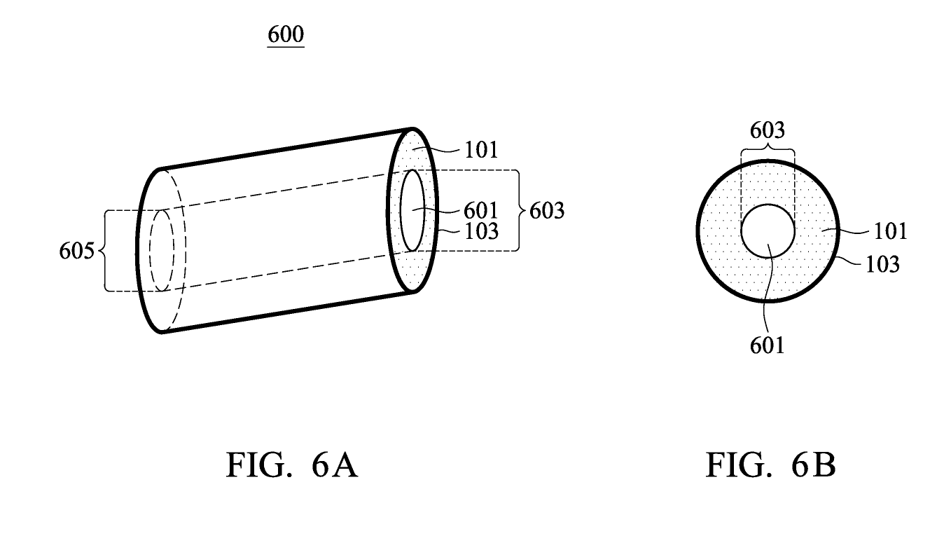

FIGS. 6A and 6B show another tube-shaped embodiment for the dual-layer composite material of the present disclosure in the form of a 3-dimensional shape comprising a hollow part or channel with at least one opening. FIG. 6A is a front view, and FIG. 6B is a cross-sectional view. In the tube-shaped dual-layer composite material comprising a hollow part or channel with at least one opening of the present disclosure 600 of FIGS. 6A and 6B, the barrier film layer 103 is located outside of the tube-shaped dual-layer composite material of the present disclosure while the porous scaffold layer 101 is located inside of the tube-shaped dual-layer composite material of the present disclosure 600 and encompasses the channel 601, the opening 603, and the opening 605.

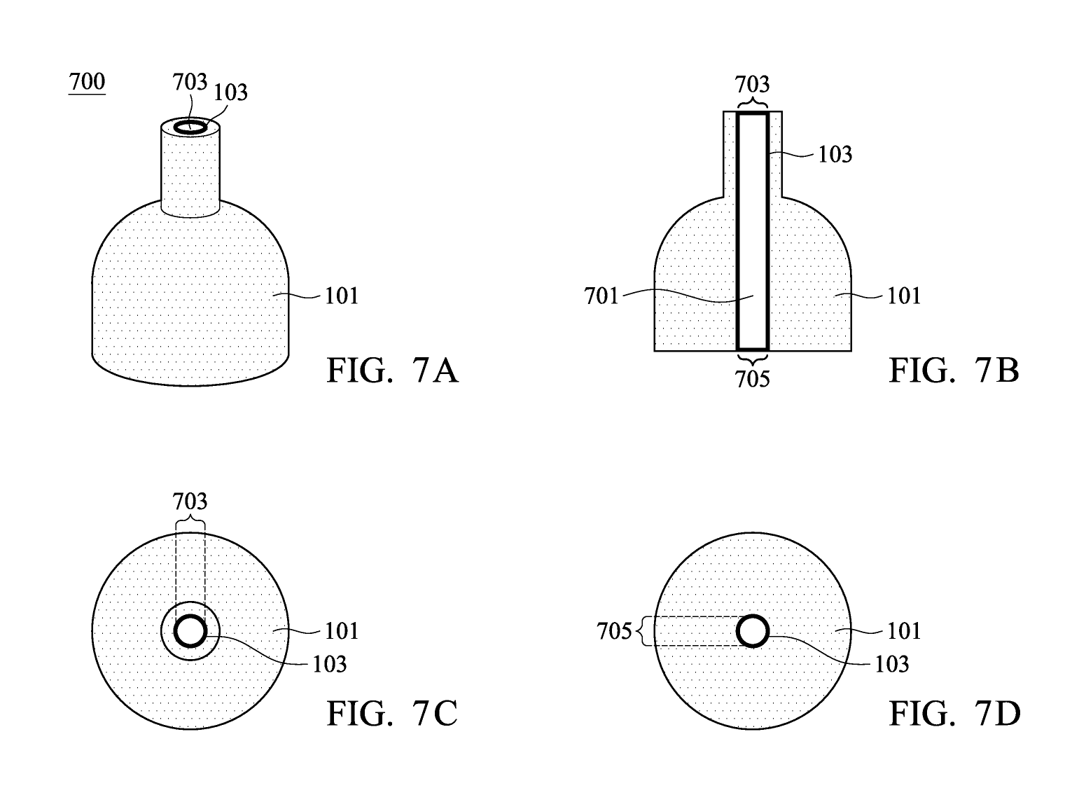

FIGS. 7A to 7D show a funnel-shaped embodiment for the dual-layer composite material of the present disclosure in the form of a 3-dimensional shape comprising a hollow part or channel with at least one opening. FIG. 7A is a front view, FIG. 7B is a longitudinal section view, FIG. 7C is a top view and FIG. 7D is a bottom view. In the funnel-shaped dual-layer composite material comprising a hollow part or channel with at least one opening of the present disclosure 700 of FIGS. 7A to 7D, the porous scaffold layer 101 is located outside of the funnel-shaped dual-layer composite material of the present disclosure while the barrier film layer 103 is located inside of of the funnel-shaped dual-layer composite material of the present disclosure 700 and encompasses the channel 701, the opening 703, and the opening 705. In one embodiment, the funnel-shaped dual-layer composite material comprising a hollow part or channel with at least one opening of the present disclosure 700 can be applied to a postsurgical wound treatment for a uterine cervix operation.

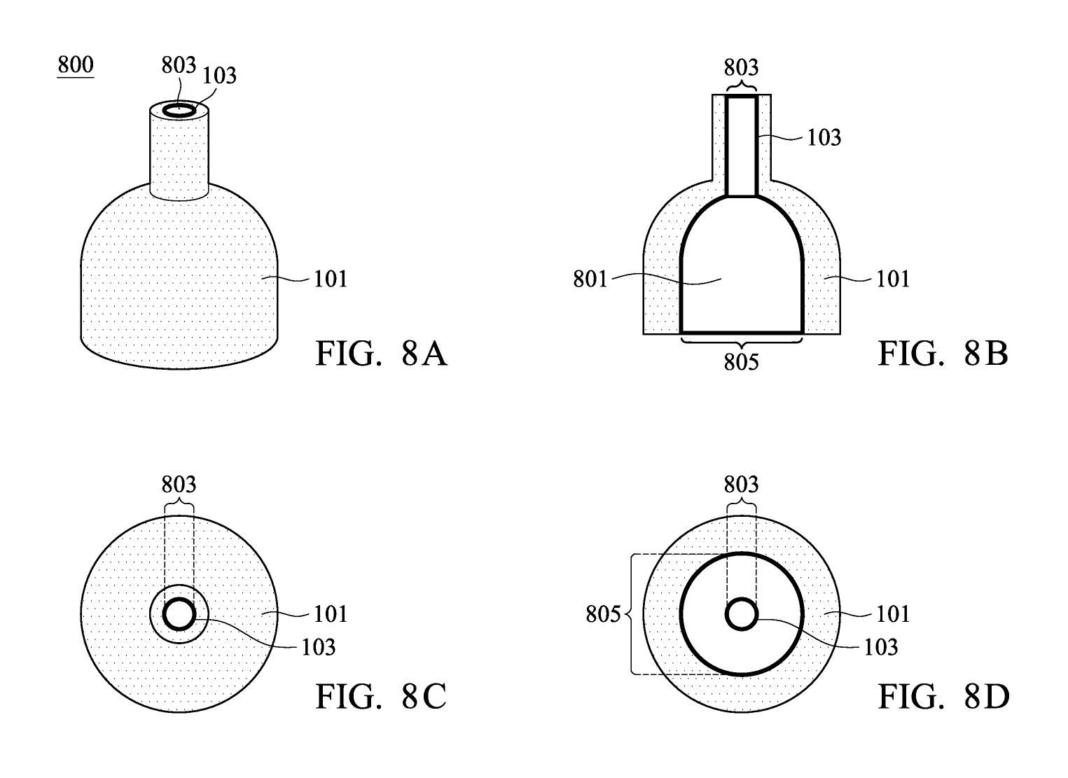

FIGS. 8A to 8D show another funnel-shaped embodiment for the dual-layer composite material of the present disclosure in the form of a 3-dimensional shape comprising a hollow part or channel with at least one opening. FIG. 8A is a front view, FIG. 8B is a longitudinal section view, FIG. 8C is a top view and FIG. 8D is a bottom view. In the funnel-shaped dual-layer composite material comprising a hollow part or channel with at least one opening of the present disclosure 800 of FIGS. 8A to 8D, the porous scaffold layer 101 is located outside of the funnel-shaped dual-layer composite material of the present disclosure while the barrier film layer 103 is located inside of the funnel-shaped dual-layer composite material of the present disclosure 800 and encompasses the funnel-shaped hollow part 801, the opening 803 at the neck of the funnel-shaped hollow part, and the opening 805 at the bottom of the funnel-shaped hollow part. In one embodiment, the funnel-shaped dual-layer composite material comprising a hollow part or channel with at least one opening of the present disclosure 800 can be applied to a postsurgical wound treatment for a uterine cervix operation.

In another embodiment, the present disclosure further provides bio-medical equipment which comprises the dual-layer composite material formed by the method for forming a dual-layer composite material of the present disclosure mentioned above.

In addition, in one embodiment, the present disclosure also provides a dual-layer composite material.

The foregoing dual-layer composite material of the present disclosure may comprise, but is not limited to, a porous scaffold layer and a barrier film layer formed on a surface of the porous scaffold layer, wherein the porous scaffold layer and the barrier film layer are inseparable from each other.

The thickness of the porous scaffold layer mentioned above may be about 0.5-5.0 mm, but is not limited thereto. Furthermore, the thickness of the barrier film layer may be about 0.05-0.5 mm, but is not limited thereto.

The porous scaffold layer of the dual-layer composite material of the present disclosure has the effect of accelerating wound-healing and/or tissue regeneration. Moreover, the porous scaffold layer of the dual-layer composite material of the present disclosure can be degraded naturally after the wound has healed. The foregoing porous scaffold layer is a scaffold structure, and a pore size of the porous scaffold layer may be about 100-500 μm, but is not limited thereto.

The barrier film layer of the dual-layer composite material of the present disclosure mentioned above has the effect of preventing tissue-adhesion. The foregoing barrier film layer is a non-porous layer, and the barrier film layer has the property of separating from the injury site and the function of providing a proper interface between the wound and other areas, attenuating the inflammatory signaling, etc., but is not limited thereto. When the dual-layer composite material of the present disclosure is applied to a wound, the barrier film layer will become a gel within 24 to 48 hours, and can be slowly resorbed and excreted from the body in less than 28 days.

The preceding porous scaffold layer may be formed by at least one first biodegradable polymer. Examples of at least one first biodegradable polymer which is suitable for forming the porous scaffold layer mentioned above may include, but are not limited to, collagen, gelatin, chitosan, a combination of collagen, gelatin or chitosan, and hyaluronic acid, and so on. In one embodiment, the at least one first biodegradable polymer may be collagen, such as type I collagen. Moreover, in another embodiment, the at least one first biodegradable polymer may be a combination of collagen and hyaluronic acid.

Moreover, the preceding barrier film layer may be formed by a second biodegradable polymer. Examples of second biodegradable polymer which is suitable for forming the barrier film layer mentioned above may include collagen, gelatin, chitosan, hyaluronic acid, etc., but are not limited thereto.

The dual-layer composite material of the present disclosure may be in the form of a membrane or in the form of a 3-dimensional shape (for example, referring to FIGS. 2 to 7D), but it is not limited thereto.

In further another embodiment, the present disclosure further provides bio-medical equipment comprising the foregoing dual-layer composite material.

### EXAMPLES

### A. Preparation of Different Dual-Layer Composite Materials of the Present Disclosure

### Example 1

### Preparation of a Dual-Layer Composite Material Having a Porous Collagen Scaffold Layer and a Hyaluronic Acid Barrier Film Layer

The method for preparing a dual-layer composite material having a porous collagen scaffold layer and a hyaluronic acid barrier film layer is described below.

1. 4.5 g type I collagen was added into the blender containing 300 ml 10% isopropyl Alcohol, then was blended under 5.0° C., under 10500 rpm for 90 minutes to obtain a slurry.

2. The slurry was placed into a vacuum oven to be vacuumed to remove the gas in the slurry.

3. 10 ml of the slurry was poured into a mold with the dimension of 6×6 cm² and placed in a freeze-dryer to be frozen at −30° C.

4. The vacuum pump of the freeze-dryer was started to keep the pressure of the freeze-dryer chamber under 200 mTorr.

5. The slurry in the freeze-dryer was maintained at −30° C. for 3 hours.

6. The temperature of the freeze-dryer was raised to 0° C. and the slurry was maintained at this temperature for 12 hours.

7. The temperature of the freeze-dryer was raised to 30° C. and the slurry was maintained at this temperature for 6 hours.

8. The vacuum pump of the freeze-dryer was stopped and the formed scaffold body was taken out of the vacuum chamber.

9. The scaffold body was placed in a the vacuum oven and the vacuum pump of was started to keep the pressure under 200 mTorr, and then the temperature of the vacuum oven was raised to 105° C. to maintain the scaffold body at this temperature for 24 hours.

10. 30 ml of water and 2.0 g of hyaluronic acid were added into a 50-ml syringe, and then left to stand overnight to form a gel.

11. Water was added into the syringe to the total volume of the content in the syringe to be 50 ml.

12. The formed hyaluronic acid gel was stirred tenderly to a homogeneous stage.

13. The syringe was vertically placed to let the bubbles depart from the hyaluronic acid gel surface.

14. 20 ml of the hyaluronic acid gel was added into a plastic mold with the dimension of 6×6 cm² and the surface of the hyaluronic acid gel waited to be flattened.

15. The scaffold body was placed onto the surface of the hyaluronic acid gel and air-dried under room temperature for 24 hours in a laminar flow.

### Example 2

### Preparation of a Dual-Layer Composite Material Having a Porous Collagen/Hyaluronic Acid Scaffold Layer and a Collagen Barrier Film Layer

The method for preparing a dual-layer composite material having a porous collagen/hyaluronic acid scaffold layer and a collagen barrier film layer is described below.

1. 4.5 g type I collagen was added into the blender containing 250 ml 0.05M acetic acid, then was blended at 5.0° C., under 10500 rpm for 90 minutes to obtain a collagen slurry.

2. 0.15 g hyaluronic acid was added into 50 ml of 0.05M acetic acid and stirred to be well-dissolved.

3. The hyaluronic acid solution was added into the blender containing the foregoing collagen slurry and blended at 5.0° C., under 10500 rpm for 90 minutes to obtain a collagen/hyaluronic acid slurry.

4. The collagen/hyaluronic acid slurry was placed into a vacuum oven to be vacuumed to remove the gas in the collagen/hyaluronic acid slurry.

5. 10 ml of the collagen/hyaluronic acid slurry was poured into a mold with the dimension of 6×6 cm² and placed in a freeze-dryer to be frozen at −30° C.

6. The vacuum pump of the freeze-dryer was started to keep the pressure of the freeze-dryer chamber under 200 mTorr.

7. The slurry in the freeze-dryer was maintained at −30° C. for 3 hours.

8. The temperature of the freeze-dryer was raised to 0° C. and the slurry was maintained at this temperature for 12 hours.

9. The temperature of the freeze-dryer was raised to 30° C. and the slurry was maintained at this temperature for 4 hours.

10. The vacuum pump of the freeze-dryer was stopped and the formed collagen/hyaluronic acid scaffold body was taken out of the vacuum chamber.

11. The collagen/hyaluronic acid scaffold body was placed in a the vacuum oven and the vacuum pump of was started to keep the pressure under 200 mTorr, and then the temperature of the vacuum oven was raised to 105° C. to maintain the scaffold body at this temperature for 24 hours.

12. 30 ml of 0.5 M acetic acid and 4.0 g of collagen were added into a 50-ml syringe, and then left to stand overnight to form a gel.

13. 0.5 M acetic acid was added into the syringe to the total volume of the content in the syringe to be 50 ml.

14. The formed collagen gel was stirred tenderly to a homogeneous stage.

15. The syringe was vertically placed to let the bubbles depart from the collagen gel surface.

16. 20 ml of the collagen gel was added into a plastic mold with the dimension of 6×6 cm² and the surface of the collagen gel waited to be flattened.

17. The collagen/hyaluronic acid scaffold body was placed onto the surface of the collagen gel and air-dried under room temperature for 24 hours in a laminar flow.

### B. Animal Experiments

(a) Material and Method

1. Electronic equipment: weight scale, electric razor, electro-coagulator (60 W)

2. Anesthetic: Zoletil 50: 0.025 ml/100 g body weight (B.W.)+xylazine (Rompun): 0.025 ml/100 g body weight (SD Rat)

3. Solution: ethanol, sterile saline solution, tincture of iodine, formalin fixing solution.

4. General material: paper towel, 50 ml centrifuge tube, plastic dropper, gauze, trash bag, 1 ml-syringe

5. Suture: 3-0 black silk sutures

Surgical instrument: surgical scissors, dressing forceps with single hook, needle holders, hemostatic forceps, scalpel

(b) Experimental Animals

1. Rat

Strain: Sprague-Dawley (SD) Rats

Sex: Female

Age: 12-14 weeks

Body weight: 210-260 g

(c) Experimental Methods and Results

1. Method

1.1 The rats were divided into three groups, wherein the first group was a control group, the second group was treated with a commercial product, Seprafilm®, and the third group was treated with a dual-layer composite material of the present disclosure (the dual-layer composite material formed by Example 1 shown above).

Or, the rats were divided into four groups, wherein the first group was a control group, the second group was treated with a commercial product, Seprafilm®, and the third group and the fourth group both were treated with a dual-layer composite material of the present disclosure (the dual-layer composite material formed by Example 1 shown above).

Or, the rats were divided into six groups, wherein the first group, the second group, and the third group were control groups, and the fourth group, the fifth group, and the sixth group were all treated with a dual-layer composite material of the present disclosure (the dual-layer composite material formed by Example 2 shown above).

1.2 Anesthesia

Zoletil 50: 0.025 ml/100 g body weight (B.W.)+xylazine (Rompun): 0.025 ml/100 g body weight; intra-peritoneal (I.P.) injection

1.3 Shaving Hairs and Spraying Ethanol

Hairs on the abdomen were shaved by an electric razor, and then the ethanol was sprayed thereon to perform a local sterilization to prevent infection.

1.4 Laparotomy

Abdomen of rat was cut upward from the second pair of teats near the anus to create a wound with a size of 3-4 cm

1.5 Creating Wounds

1.5.1 Stitch Buttons on Right Peritoneal Wall

Stitch buttons were stitched on right peritoneal wall from the position corresponding to the second pair of teats for making a total of four buttons. The buttons were spaced at an interval of 0.5 cm. Each button has three knots thereon.

1.5.2 Incisions and Sutures for Left Uterine Horn

A 1 cm wound in the uterine horn near a position 1 cm above the uterus was cut and then sutured with three sutures.

1.5.3 Wound on Left Peritoneal Wall

A wound with a diameter of about 2 cm and a depth of about 0.5-1 mm was made in the left peritoneal wall.

1.6 Use of Adhesion Prevention Material

After the redundant liquid was removed from the wound, a adhesion prevention membrane with a size of 2 cm×2 cm (Seprafilm® or a dual-layer composite material of the present disclosure) was applied by dry forceps to cover the wound (there was no cover for the control group). The adhesion prevention membrane had to completely cover the wound to provide complete protection.

1.7 Suture for Wound

Suture was performed by 3-0 black silk sutures. The muscle layer was sutured by continuous suture while the epidermal layer was sutured by non-continuous suture. Finally, tincture of iodine was smeared on the wound on the epidermal layer, and the rat waited to be revived.

1.8 Evaluation of Efficacy of Adhesion Prevention Membranes

14 days after the operation, post-operation adhesion was evaluated by operators with double check, and adhesion evaluation table was made according to Linsky scoring system (see J. Reprod. Med., 32, 17-20, 1987 and Human Reproduction Vol. 18, No. 8 pp. 1703-1706, 2003 which are incorporated herein by reference). Also, the tissue specimens were collected for pathological evaluation of tissue repair.

Scoring standard for the Linsky scoring system is described below

(1) Adhesion Extent

0=no adhesions

1=20% of the traumatized area

2=50% of the traumatized area

3=total involvement

(2) Adhesion Severity

0=no resistance for separation

0.5=some resistance for separation (moderate force is required)

1=sharp dissection is needed

1.9 Notice

1.9.1 Treatment time for each rat must be the same (25-30 minutes/rat)

1.9.2 All operations have to be performed by the same person to ensure consistency.

2. Experimental Results

(1) Animal Experiments for the Material from Example 1

Three independent experiments were performed.

(i) First Experiment

Positions and manner for creating wounds in the first experiment comprised stitch buttons on right peritoneal wall, electro-coagulation on right uterine horn and incisions and sutures for left uterine horn. After the operations, the rats were observed for 28 days, and 14 days after the operations, wound adhesion evaluation was performed on the rats in each group. The rat numbered 1-1 died after the operations. Evaluation results for wound adhesion for each group are shown in Table 1. According to Table 1, as compared to the commercial product, the dual-layer composite material of the present disclosure has a better anti-adhesion effect.

TABLE 1
Adhesion evaluation for the first experiment

| | Stitch buttons | | | Electro- | | | Incisions and | | | | |
|---|---|---|---|---|---|---|---|---|---|---|---|
| | on right | | | coagulation on | | | sutures for | | | Body weight (g) | |
| | peritoneal wall | | | right uterine horn | | | left uterine horn | | | Before | After |
| Rat number | E | S | Total | E | S | Total | E | S | Total | operations | operations |

First group (control group)

| | | | | | | | | | | | |
|---|---|---|---|---|---|---|---|---|---|---|---|
| 1-2 | 1.00 | 0.50 | 1.50 | 0.00 | 0.00 | 0.00 | 3.00 | 1.00 | 4.00 | 240.00 | 250.00 |
| 1-3 | 3.00 | 1.00 | 4.00 | 3.00 | 1.00 | 4.00 | 3.00 | 1.00 | 4.00 | 240.00 | 271.00 |
| 1-4 | 3.00 | 1.00 | 4.00 | 3.00 | 1.00 | 4.00 | 3.00 | 1.00 | 4.00 | 240.00 | 284.00 |
| Mean | 2.33 | 0.83 | 3.17 | 2.00 | 0.67 | 2.67 | 3.00 | 1.00 | 4.00 | 240.00 | 268.33 |
| Standard | 0.94 | 0.24 | 1.18 | 1.41 | 0.47 | 1.89 | 0.00 | 0.00 | 0.00 | 0.00 | 14.01 |
| deviation | | | | | | | | | | | |

Second group (Seprafilm ®)

| | | | | | | | | | | | |
|---|---|---|---|---|---|---|---|---|---|---|---|
| 2-1 | 0.00 | 0.00 | 0.00 | 2.00 | 0.50 | 2.50 | 2.00 | 1.00 | 3.00 | 240.00 | 282.00 |
| 2-2 | 3.00 | 1.00 | 4.00 | 3.00 | 1.00 | 4.00 | 3.00 | 1.00 | 4.00 | 240.00 | 266.00 |
| 2-3 | 2.00 | 0.50 | 2.50 | 2.00 | 0.50 | 2.50 | 2.00 | 1.00 | 3.00 | 240.00 | 277.00 |
| Mean | 1.67 | 0.50 | 2.17 | 2.33 | 0.67 | 3.00 | 2.33 | 1.00 | 3.33 | 240.00 | 275.00 |
| Standard | 1.25 | 0.41 | 1.65 | 0.47 | 0.24 | 0.71 | 0.47 | 0.00 | 0.47 | 0.00 | 6.68 |
| deviation | | | | | | | | | | | |

Third group (Example 1)

| | | | | | | | | | | | |
|---|---|---|---|---|---|---|---|---|---|---|---|
| 3-1 | 2.00 | 0.50 | 2.50 | 1.00 | 0.00 | 1.00 | 2.00 | 1.00 | 3.00 | 240.00 | 281.00 |
| 3-2 | 1.00 | 0.50 | 1.50 | 2.00 | 0.50 | 2.50 | 3.00 | 1.00 | 4.00 | 240.00 | 283.00 |
| 3-3 | 2.00 | 1.00 | 3.00 | 0.00 | 0.00 | 0.00 | 2.00 | 1.00 | 3.00 | 240.00 | 272.00 |
| Mean | 1.67 | 0.67 | 2.33 | 1.00 | 0.17 | 1.17 | 2.33 | 1.00 | 3.33 | 240.00 | 278.67 |
| Standard | 0.47 | 0.24 | 0.62 | 0.82 | 0.24 | 1.03 | 0.47 | 0.00 | 0.47 | 0.00 | 4.78 |
| deviation | | | | | | | | | | | |

E: Adhesion extent
S: Adhesion severity

(ii) Second Experiment

Positions and manner for creating wounds in the second experiment comprised stitch buttons on right peritoneal wall, electro-coagulation on left peritoneal wall and incisions and sutures for left uterine horn. After operations, the rats were observed for 14 days, and 14 days after operations, wound adhesion evaluation was performed on the rats in each group. The rat numbered 3-1 died after the operations. Evaluation results for wound adhesion for each group are shown in Table 2. According to Table 2, as compared to the commercial product, the dual-layer composite material of the present disclosure has a better anti-adhesion effect.

TABLE 2
Adhesion evaluation for the second experiment

| | Stitch buttons | | | Electro- | | | Incisions and | | | | |
|---|---|---|---|---|---|---|---|---|---|---|---|
| | on right | | | coagulation on | | | sutures | | | Body weight (g) | |
| | peritoneal wall | | | left peritoneal wall | | | for left uterine horn | | | Before | After |
| Rat number | E | S | Total | E | S | Total | E | S | Total | operations | operations |

First group (control group)

| | | | | | | | | | | | |
|---|---|---|---|---|---|---|---|---|---|---|---|
| 1-1 | 3.00 | 1.00 | 4.00 | 2.00 | 0.50 | 2.50 | 3.00 | 1.00 | 4.00 | 250.00 | 260.50 |
| 1-2 | 3.00 | 1.00 | 4.00 | 0.00 | 0.00 | 0.00 | 3.00 | 1.00 | 4.00 | 248.20 | 265.00 |
| 1-3 | 3.00 | 1.00 | 4.00 | 0.00 | 0.00 | 0.00 | 3.00 | 1.00 | 4.00 | 251.70 | 265.00 |
| Mean | 3.00 | 1.00 | 4.00 | 0.67 | 0.17 | 0.83 | 3.00 | 1.00 | 4.00 | 249.97 | 263.50 |
| Standard | 0.00 | 0.00 | 0.00 | 0.94 | 0.24 | 1.18 | 0.00 | 0.00 | 0.00 | 1.43 | 2.12 |
| deviation | | | | | | | | | | | |

Second group (Seprafilm ®)

| | | | | | | | | | | | |
|---|---|---|---|---|---|---|---|---|---|---|---|
| 2-1 | 3.00 | 1.00 | 4.00 | 0.00 | 0.00 | 0.00 | 2.00 | 1.00 | 3.00 | 250.00 | 289.00 |
| 2-2 | 3.00 | 1.00 | 4.00 | 0.00 | 0.00 | 0.00 | 2.00 | 0.50 | 2.50 | 246.80 | 267.00 |
| 2-3 | 3.00 | 0.50 | 3.50 | 0.00 | 0.00 | 0.00 | 3.00 | 1.00 | 4.00 | 266.10 | 275.00 |
| Mean | 3.00 | 0.83 | 3.83 | 0.00 | 0.00 | 0.00 | 2.33 | 0.83 | 3.17 | 254.30 | 277.00 |
| Standard | 0.00 | 0.24 | 0.24 | 0.00 | 0.00 | 0.00 | 0.47 | 0.24 | 0.62 | 8.45 | 9.09 |
| deviation | | | | | | | | | | | |

Third group (Example 1)

| 3-1 | Dead | | | | | | | | | | |
|---|---|---|---|---|---|---|---|---|---|---|---|
| 3-2 | 2.00 | 0.50 | 2.50 | 0.00 | 0.00 | 0.00 | 2.00 | 1.00 | 3.00 | 249.10 | 253.00 |
| 3-3 | 1.00 | 0.50 | 1.50 | 0.00 | 0.00 | 0.00 | 2.00 | 1.00 | 3.00 | 264.60 | 254.00 |
| Mean | 1.50 | 0.50 | 2.00 | 0.00 | 0.00 | 0.00 | 2.00 | 1.00 | 3.00 | 256.85 | 253.50 |
| Standard | 0.50 | 0.00 | 0.50 | 0.00 | 0.00 | 0.00 | 0.00 | 0.00 | 0.00 | 7.75 | 0.50 |
| deviation | | | | | | | | | | | |

E: Adhesion extent
S: Adhesion severity

(ii) Third Experiment

Positions and manner for creating wounds in the third experiment comprised stitch buttons on right peritoneal wall, electro-coagulation on left peritoneal wall and incisions and sutures for left uterine horn. After the operations, the rats were observed for 14 days, and 14 days after the operations, wound adhesion evaluation was performed on the rats in each group. Evaluation results for wound adhesion for each group are shown in Table 3. According to Table 3, as compared to the commercial product, the dual-layer composite material of the present disclosure has a better anti-adhesion effect.

TABLE 3
Adhesion evaluation for the third experiment

| | Stitch buttons | | | Electro- | | | Incisions | | | | |
|---|---|---|---|---|---|---|---|---|---|---|---|
| | on right | | | coagulation on | | | and sutures for | | | Body weight (g) | |
| | peritoneal wall | | | left peritoneal wall | | | left uterine horn | | | Before | After |
| Rat number | E | S | Total | E | S | Total | E | S | Total | operations | operations |

First group (control group)

| | | | | | | | | | | | |
|---|---|---|---|---|---|---|---|---|---|---|---|
| 1-1′ | 3.00 | 1.00 | 4.00 | 0.00 | 0.00 | 0.00 | 3.00 | 1.00 | 4.00 | 265.66 | 261.00 |
| 1-2′ | 3.00 | 1.00 | 4.00 | 1.00 | 0.50 | 1.50 | 3.00 | 1.00 | 4.00 | 255.00 | 281.00 |
| 1-3′ | 3.00 | 1.00 | 4.00 | 3.00 | 1.00 | 4.00 | 3.00 | 1.00 | 4.00 | 255.00 | 265.00 |
| Mean | 3.00 | 1.00 | 4.00 | 1.33 | 0.50 | 1.83 | 3.00 | 1.00 | 4.00 | 258.55 | 269.00 |
| Standard | 0.00 | 0.00 | 0.00 | 1.25 | 0.41 | 1.65 | 0.00 | 0.00 | 0.00 | 5.03 | 8.64 |
| deviation | | | | | | | | | | | |

Second group (Seprafilm ®)

| | | | | | | | | | | | |
|---|---|---|---|---|---|---|---|---|---|---|---|
| 2-1′ | 3.00 | 1.00 | 4.00 | 0.00 | 0.00 | 0.00 | 3.00 | 1.00 | 4.00 | 263.67 | 258.00 |
| 2-2′ | 3.00 | 1.00 | 4.00 | 3.00 | 1.00 | 4.00 | 3.00 | 1.00 | 4.00 | 258.00 | 300.00 |
| 2-3′ | 3.00 | 1.00 | 4.00 | 0.00 | 0.00 | 0.00 | 3.00 | 1.00 | 4.00 | 260.00 | 286.00 |
| Mean | 3.00 | 1.00 | 4.00 | 1.00 | 0.33 | 1.33 | 3.00 | 1.00 | 4.00 | 260.56 | 281.33 |
| Standard | 0.00 | 0.00 | 0.00 | 1.41 | 0.47 | 1.89 | 0.00 | 0.00 | 0.00 | 2.35 | 17.46 |
| deviation | | | | | | | | | | | |

Third group (Example 1)

| | | | | | | | | | | | |
|---|---|---|---|---|---|---|---|---|---|---|---|
| 3-1′ | 3.00 | 0.50 | 3.50 | 0.00 | 0.00 | 0.00 | 0.00 | 0.00 | 0.00 | 266.13 | 297.00 |
| 3-2′ | 3.00 | 0.50 | 3.50 | 3.00 | 1.00 | 4.00 | 0.00 | 0.00 | 0.00 | 260.20 | 269.00 |
| 3-3′ | 3.00 | 0.50 | 3.50 | 3.00 | 1.00 | 4.00 | 0.00 | 0.00 | 0.00 | 259.60 | 284.00 |
| Mean | 3.00 | 0.50 | 3.50 | 2.00 | 0.67 | 2.67 | 0.00 | 0.00 | 0.00 | 261.98 | 283.33 |
| Standard | 0.00 | 0.00 | 0.00 | 1.41 | 0.47 | 1.89 | 0.00 | 0.00 | 0.00 | 2.95 | 11.44 |
| deviation | | | | | | | | | | | |

Fourth group (Example 1)

| | | | | | | | | | | | |
|---|---|---|---|---|---|---|---|---|---|---|---|
| 4-1′ | 3.00 | 0.50 | 3.50 | 3.00 | 1.00 | 4.00 | 0.00 | 0.00 | 0.00 | 264.47 | 291.00 |
| 4-2′ | 3.00 | 0.50 | 3.50 | 0.00 | 0.00 | 0.00 | 1.00 | 0.50 | 1.50 | 266.00 | 271.00 |
| 4-3′ | 1.00 | 0.50 | 1.50 | 1.00 | 0.50 | 1.50 | 0.00 | 0.00 | 0.00 | 262.60 | 276.00 |
| Mean | 2.33 | 0.50 | 2.83 | 1.33 | 0.50 | 1.83 | 0.33 | 0.17 | 0.50 | 264.36 | 279.33 |
| Standard | 0.94 | 0.00 | 0.94 | 1.25 | 0.41 | 1.65 | 0.47 | 0.24 | 0.71 | 1.39 | 8.50 |
| deviation | | | | | | | | | | | |

E: Adhesion extent
S: Adhesion severity

(2) Animal Experiments for the Material from Example 2

Positions and manner for creating wounds comprised electro-coagulation on left peritoneal wall and incisions and sutures for left uterine horn. After operations, the rats were observed for 14 days, and 14 days after operations, wound adhesion evaluation was performed on the rat in each group. Evaluation results for wound adhesion for each group are shown in Table 4. According to Table 4, it is known that the dual-layer composite material of the present disclosure has a better anti-adhesion effect.

TABLE 4
Adhesion evaluation

| | Electro- | | | | | |
|---|---|---|---|---|---|---|
| | coagulation | | Incisions and | | | |
| | on left | | sutures for left | | Body weight (g) | |
| | peritoneal wall | | uterine horn | | Before | After |
| | E | S | E | S | operations | operations |
| First group | 3 | 1 | 3 | 1 | 315.3 | 312.8 |
| (control group) | | | | | | |
| Second group | 3 | 1 | 3 | 1 | 311.8 | 309.3 |
| (control group) | | | | | | |
| Third group | 0 | 0 | 3 | 1 | 299.7 | 307.8 |
| (control group) | | | | | | |
| Fourth group | 0 | 0 | 2 | 0.5 | 346.1 | 342.3 |
| (Example 2) | | | | | | |
| Fifth group | 0 | 0 | 0 | 0 | 311.4 | 287.3 |
| (Example 2) | | | | | | |
| Sixth group | 1 | 0.5 | 1 | 0.5 | 289.7 | 296.5 |
| (Example 2) | | | | | | |

E: Adhesion extent
S: Adhesion severity

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method for forming a dual-layer composite material, comprising:
coating a barrier-film layer-forming material onto a surface of a porous scaffold layer to form a dual-layer intermediate product; and
drying the dual-layer intermediate product to form a dual-layer composite material which comprises the porous scaffold layer and a barrier film layer, wherein the porous scaffold layer and the barrier film layer are inseparable from each other, and
wherein a method for forming the porous scaffold layer comprises:
mixing at least one first biodegradable polymer with a first solvent under a stirring speed of 3500-12000 rpm, at about 4-10° C., for about 90-180 minutes, to form a slurry, wherein the at least one first biodegradable polymer is selected from a group consisting of collagen, gelatin, chitosan, and a combination of collagen, gelatin or chitosan, and hyaluronic acid;
placing the slurry into a mold and freezing the slurry;
performing a lyophilization procedure on the slurry to remove water molecules and obtain a scaffold body; and
performing a vacuum heating procedure on the scaffold body to dehydrate and crosslink the scaffold body to form the porous scaffold layer, and
wherein a method for forming the barrier-film layer-forming material comprises:
mixing a second biodegradable polymer with a second solvent and then letting it stand to form a gel, wherein the second biodegradable polymer is selected from a group consisting of collagen, gelatin, chitosan and hyaluronic acid; and
stirring the gel to a homogeneous stage to form the barrier-film layer-forming material.

2. The method for forming a dual layerdual-layer composite material as claimed in claim 1, wherein the at least one first biodegradable polymer accounts for about 1.0-2.0 wt % of the slurry.

3. The method for forming a dual layerdual-layer composite material as claimed in claim 1, wherein the at least one first biodegradable polymer is collagen.

4. The method for forming a dual layerdual-layer composite material as claimed in claim 3, wherein the collagen is type I collagen.

5. The method for forming a dual layerdual-layer composite material as claimed in claim 1, wherein the at least one first biodegradable polymer is a combination of collagen, gelatin or chitosan, and hyaluronic acid.

6. The method for forming a dual layerdual-layer composite material as claimed in claim 5, before the step of mixing at least one first biodegradable polymer with a first solvent under a stirring speed of 3500-12000 rpm, at about 4-10° C., for about 90-180 minutes, to form a slurry, further comprising:
mixing the collagen, gelatin or chitosan with the first solvent under a stirring speed of 3500-12000 rpm, at about 4-10° C., for about 90-180 minutes, previously, to form a mixture; and
pouring a solution of the hyaluronic acid into the mixture.

7. The method for forming a dual layerdual-layer composite material as claimed in claim 1, wherein the at least one first biodegradable polymer is a combination of collagen and hyaluronic acid.

8. The method for forming a dual layerdual-layer composite material as claimed in claim 7, wherein the collagen is type I collagen.

9. The method for forming a dual layerdual-layer composite material as claimed in claim 7, wherein a weight ratio of the collagen to the hyaluronic acid is about 90-99.9:10-0.1.

10. The method for forming a dual layerdual-layer composite material as claimed in claim 1, wherein the first solvent comprises water, isopropanol or acetic acid.

11. The method for forming a dual layerdual-layer composite material as claimed in claim 1, wherein in the step of placing the slurry into a mold and freezing the slurry, the slurry is frozen at about −40 to −20° C.

12. The method for forming a dual layerdual-layer composite material as claimed in claim 1, wherein a pressure in the lyophilization procedure is about 30-200 mTorr.

13. The method for forming a dual layerdual-layer composite material as claimed in claim 12, wherein the lyophilization procedure comprises:
at about −40 to −20° C., freezing the slurry for about 3-6 hours;
at about −40 to 10° C., freezing the slurry for about 12-16 hours; and
placing the slurry at about 20 to 30° C., for about 3-6 hours.

14. The method for forming a dual layerdual-layer composite material as claimed in claim 1, wherein a pressure in the vacuum heating procedure is about 30-200 mTorr.

15. The method for forming a dual layerdual-layer composite material as claimed in claim 1, wherein a temperature in the vacuum heating procedure is about 80-110° C.

16. The method for forming a dual layerdual-layer composite material as claimed in claim 1, wherein time for the vacuum heating procedure is about 12-24 hours.

17. The method for forming a dual layerdual-layer composite material as claimed in claim 1, between the step of mixing at least one first biodegradable polymer with a first solvent under a stirring speed of 3500-12000 rpm, at about 4-10° C., for about 90-180 minutes, to form a slurry, and the step of placing the slurry into a mold and freezing the slurry, further comprising a step of removing gas in the slurry.

18. The method for forming a dual layerdual-layer composite material as claimed in claim 1, wherein the second biodegradable polymer accounts for about 3-10% w/v of the gel.

19. The method for forming a dual layerdual-layer composite material as claimed in claim 1, wherein the second biodegradable polymer is hyaluronic acid.

20. The method for forming a dual layerdual-layer composite material as claimed in claim 1, wherein the second biodegradable polymer is collagen.

21. The method for forming a dual layerdual-layer composite material as claimed in claim 20, wherein the collagen is type I collagen.

22. The method for forming a dual layerdual-layer composite material as claimed in claim 1, wherein the second solvent comprises water or acetic acid.

23. The method for forming a dual layerdual-layer composite material as claimed in claim 1, wherein in the step of mixing a second biodegradable polymer with a second solvent and then letting it stand, with a standing time of about 12-24 hours.

24. The method for forming a dual-layer composite material as claimed in claim 1, after the step of stirring the gel to a homogeneous stage, further comprising removing bubbles in the gel.

25. The method for forming a dual-layer composite material as claimed in claim 1, wherein the dual-layer intermediate product is dried at about 15-30° C.

26. The method for forming a dual-layer composite material as claimed in claim 1, wherein the dual-layer intermediate product is dried for about 12-24 hours.

27. The method for forming a dual-layer composite material as claimed in claim 1, wherein the dual-layer intermediate product is dried at a room temperature for about 24 hours.

28. A dual-layer composite material formed by the method for forming a dual-layer composite material as claimed in claim 1, wherein the dual-layer composite material is in the form of a membrane or in the form of a 3-dimensional shape comprising a hollow part or channel with at least one opening.

29. The dual-layer composite material as claimed in claim 28, wherein the 3-dimensional shape is a globe shape, an oval-shape, a tube shape, a funnel shape or a cone shape.

30. The dual-layer composite material as claimed in claim 28, wherein the dual-layer composite material is in the form of a 3-dimensional shape comprising a hollow part or channel with at least one opening, and wherein the porous scaffold layer is located outside of the 3-dimensional shape while the barrier film layer is located inside of the 3-dimensional shape and encompasses the hollow part or channel, or wherein the barrier film layer is located outside of the 3-dimensional shape while the porous scaffold layer is located inside of the 3-dimensional shape and encompasses the hollow part or channel.
